(19) United States
(12) Patent Application Publication
(10) Pub. No.: US 2014/0221711 A1
(43) Pub. Date: Aug. 7, 2014

(54) System and Process for Converting Natural Gas Into Saturated, Cyclic Hydrocarbons

(71) Applicant: Ceramatec, Inc., Salt Lake City, UT (US)

(72) Inventor: Pallavi Chitta, West Valley City, UT (US)

(73) Assignee: Ceramatec, Inc., Salt Lake City, UT (US)

(21) Appl. No.: 14/248,994

(22) Filed: Apr. 9, 2014

Related U.S. Application Data
(63) Continuation-in-part of application No. 14/090,776, filed on Nov. 26, 2013.
(60) Provisional application No. 61/809,914, filed on Apr. 9, 2013.
(60) Provisional application No. 61/731,397, filed on Nov. 29, 2012.

(51) Int. Cl. C07C 5/10; C07C 2/76

(52) U.S. Cl. CPC C07C 5/10; C07C 2/76; C07C 2523/755; C07C 2523/75
USPC 585/254; 422/187

(57) ABSTRACT

A system and process to make cyclic, saturated hydrocarbons from aromatic hydrocarbon intermediates from catalyzed nonoxidative dehydroaromatization (DHA) of methane. The system includes two reaction zones, one containing a dehydroaromatization catalyst and a second containing a hydrogenation catalyst. Methane reacts in the first reaction zone with the DHA catalyst resulting in aromatic hydrocarbons concomitantly produced with hydrogen gas. The hydrogen gas is removed and introduced to the second reaction zone with the aromatic hydrocarbon to reductively produce saturated, cyclic hydrocarbons.

### CROSS-REFERENCE TO RELATED APPLICATIONS

This application claims the benefit of U.S. application Ser. No. 14/090,776 filed Nov. 26, 2013, which claims priority to U.S. Provisional Patent Application No. 61/731,397, filed Nov. 29, 2012; and U.S. Provisional Patent Application No. 61/809,914, filed Apr. 9, 2013. The foregoing applications are incorporated by reference in the entirety.

### FIELD OF THE INVENTION

The present invention relates to method for the production of saturated, cyclic hydrocarbons from methane. More particularly, the present invention produces aromatic intermediates via dehydroaromatization (DHA) of methane followed by hydrogenation of the intermediates to form saturated, cyclic hydrocarbons such as cyclohexane and decalin.

### BACKGROUND OF THE INVENTION

Currently, petroleum crude costs six to eight times more than natural gas on an energy content basis. Moreover, approximately 97% of natural gas is currently produced from domestic sources, whereas more than 50% of the crude oil demand is imported. This presents opportunities for reduction in petroleum crude usage and has led to the emergence of new processes with more attractive economics for producing value-added chemicals and fuels from natural gas.

Benzene, which is currently produced from crude oil, is a chemical of great industrial importance with current global consumption in excess of 30 million metric tons per annum and net growth of 4% annually, leading to a total market size of more than $50,000,000,000. It is a starting material for nylons, polycarbonates, polystyrene and epoxy resins. Also, benzene can be directly converted to aniline, chlorobenzene, maleic anhydride, succinic acid, and countless other useful industrial chemicals. Benzene is a gasoline component and can be converted to cyclohexane, another gasoline component via a commercial process.

Benzene can be synthesized from natural gas (methane) using a catalyst in a single step via dehydroaromatization (DHA) route in the absence of oxygen as follows:

6CH₄→C₆H₆+9H₂

While the DHA process is commercially very attractive, there are two primary technical commercialization challenges for this reaction:
Kinetic: As hydrogen is removed, a coking reaction on the catalyst surface competes with the desired DHA reaction; and
Thermodynamic: Equilibrium conversion of methane to benzene is limited to about 12% at 700° C. and 1 atmosphere.

There is a need in the art for further advances in the DHA process which overcome the kinetic and thermodynamic challenges and which improve the yield of benzene and which limit coking of the catalyst.

In addition, cyclohexane and decalin are cyclic hydrocarbons of great industrial importance. Cyclohexane is the raw material for production of adipic acid and caprolactum at industrial scales, which are further used to produce Nylon 6 and Nylon 66 respectively. Cyclohexane is also used as a solvent for a variety of applications and is a gasoline component. Decalin is used as a solvent for a number of industrial applications.

Cyclohexane is produced industrially via hydrogenation of benzene, wherein the benzene is produced in turn via catalytic reforming of light naphtha, toluene hydrodealkylation, or steam cracking of heavy naphtha. The primary raw material for all of these processes is petroleum crude. Thus, all of the cyclohexane is currently being produced from petroleum crude. Decalin is produced from hydrogenation of naphthalene, which is in turn produced from coal tar or petroleum.

There is a significant need to produce cyclohexane from a non-petroleum crude based feedstock. As mentioned above, petroleum crude is a very expensive feedstock. Further, the total crude production is on the decline and thus unable to keep up with the net demand for it as feedstock. An alternative feedstock for production of these cyclic compounds is, therefore, needed.

As a natural by-product of the petroleum extraction process, trillions of cubic feet of natural gas are burned off, or “flared,” each year because natural gas can be expensive to store and transport for later use. This practice is both harmful to the environment and inefficient—by some accounts, the amount of natural gas flared each year is equivalent to 20% of US electricity generation. Also, due to recent developments in hydraulic fracturing, there is an abundant supply of natural gas in the United States thereby having a price point significantly lower than that of petroleum crude on an energy equivalency basis. For example natural gas costs have been as low as $2 per MMBTU in recent past, which corresponds to $12 per barrel equivalent. Petroleum crude is 6-8 times more expensive per barrel. There is a need to develop technologies to capture this natural gas for use as additional energy sources or to convert into useful chemicals that can be utilized by other industrial markets. A potential process to produce cyclic hydrocarbons from natural gas would be desirable.

### BRIEF SUMMARY OF THE INVENTION

In one aspect, an apparatus to produce one or more cyclic, saturated hydrocarbons, includes: a first reaction zone comprising a dehydroaromatization catalyst; a second reaction zone comprising a hydrogenation catalyst; a first inlet that provides a reactant hydrocarbon to the first reaction zone; a heater for heating the first reaction zone; a hydrocarbon transfer conduit located between the first and second reaction zones; a hydrogen transfer conduit located between the first and second reaction zones; and a hydrogen separation membrane disposed between the first reaction zone and the hydrogen transfer conduit.

In another aspect, a method of preparing one or more cyclic, saturated hydrocarbons, includes: contacting a reactant hydrocarbon with a dehydroaromatization catalyst in a first reaction zone to produce one or more aromatic intermediates; heating the first reaction zone; removing hydrogen from the first reaction zone through a hydrogen separation membrane; transferring the aromatic intermediate from the first reaction zone to the second reaction zone; providing the separated hydrogen to the second reaction zone; and contacting the aromatic intermediate with a hydrogenation catalyst in a second reaction zone resulting in one or more cyclic, saturated hydrocarbons.

In some embodiments, the first and second reaction zones are located in a single reactor. In some embodiments, the first and second reaction zones are located in separate reactors.

In some embodiments, the hydrogenation catalyst is nickel or cobalt.

In some embodiments, the hydrogen separation membrane comprises a ceramic membrane that selectively transports H⁺ ions at dehydroaromatization operating temperatures. In some embodiments, the hydrogen separation membrane selectively transports H⁺ ions under a hydrogen partial pressure gradient, a concentration gradient, or an applied voltage. In some embodiments, the hydrogen separation membrane comprises a perovskite, a doped cerate, a doped zirconate, or an acidic phosphate. In some embodiments, the hydrogen separation membrane comprises a Ba-cerate ceramic composite.

In some embodiments, the apparatus includes a hydrocarbon separator capable of separating reactant hydrocarbon from the first reaction zone. In some embodiments, the apparatus includes a vacuum means operably connected to the hydrogen separation membrane and hydrogen transfer conduit.

In some embodiments, the reactant hydrocarbon is methane.

In some embodiments, the method includes periodically regenerating the dehydroaromatization catalyst by contacting the dehydroaromatization catalyst with hydrogen. In some embodiments, the one or more cyclic, hydrocarbons includes decalin. In some embodiments, the one or more cyclic, hydrocarbons includes cyclohexane.

### BRIEF DESCRIPTION OF THE DRAWINGS

In order that the manner in which the above-recited and other features and advantages of the invention are obtained and will be readily understood, a more particular description of the invention briefly described above will be rendered by reference to specific embodiments thereof that are illustrated in the appended drawings. Understanding that the drawings are not made to scale, depict only some representative embodiments of the invention, and are not therefore to be considered to be limiting of its scope, the invention will be described and explained with additional specificity and detail through the use of the accompanying drawings in which:

FIG. 1 depicts the combination of a hydrogen separation membrane and a dehydroaromatization catalyst usable in the disclosed system and process.

FIG. 2 depicts a system for efficient dehydroaromatization of a reactant composition to produce an aromatic hydrocarbon using a combination of a hydrogen separation membrane and a dehydroaromatization catalyst.

FIG. 3 depicts another system for efficient dehydroaromatization of a reactant composition to produce an aromatic hydrocarbon using a combination of a hydrogen separation membrane and a dehydroaromatization catalyst.

FIG. 4 is a schematic representation of a system for dehydroaromatization of a reactant composition to produce an aromatic hydrocarbon.

FIG. 5 is schematic representation of a CHEMKIN® chemical model and simulation methodology for the disclosed system and process.

FIG. 6 is a graph of experimental results compared with chemical model simulated product selectivities as a function of methane conversion.

FIG. 7 is a conceptual model of six equilibrium reactors used by chemical process simulation software to evaluate methane conversion as a function of percentage hydrogen removal.

FIG. 8 is a graph showing the results of the chemical process simulation evaluating methane conversion as a function of percentage hydrogen removal.

FIG. 9 depicts a system for producing saturated, cyclic hydrocarbons using an embodiment of the invention.

FIG. 10 is a graph showing the results of a chemical process simulation for conversion of methane to aromatic hydrocarbon intermediates with benzene selectivity and yield as a function of hydrogen removal in an embodiment of the invention.

### DETAILED DESCRIPTION OF THE INVENTION

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment. Additionally, while the following description refers to several embodiments and examples of the various components and aspects of the described invention, all of the described embodiments and examples are to be considered, in all respects, as illustrative only and not as being limiting in any manner.

Furthermore, the described features, structures, or characteristics of the invention may be combined in any suitable manner in one or more embodiments. In the following description, numerous specific details are provided, such as examples of suitable dehydroaromatization catalysts, hydrogen separation membrane materials, operating conditions and variations, etc., to provide a thorough understanding of embodiments of the invention. One having ordinary skill in the relevant art will recognize, however, that the invention may be practiced without one or more of the specific details, or with other processes, components, materials, and so forth. In other instances, well-known structures, materials, or operations are not shown or described in detail to avoid obscuring aspects of the invention.

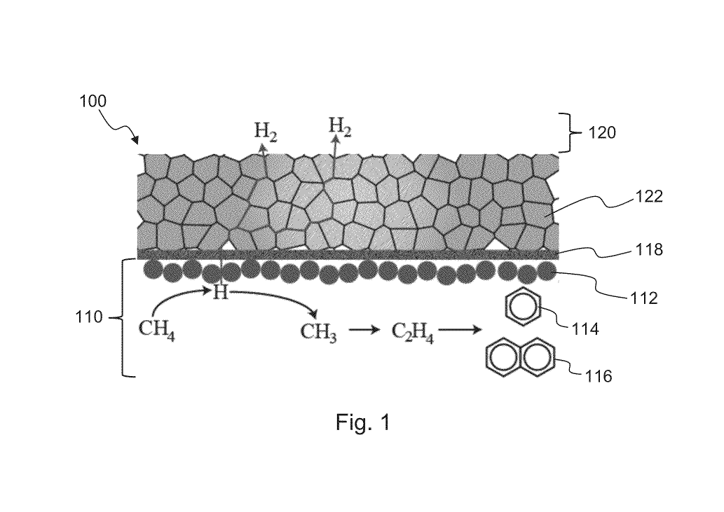

A system and process to produce an aromatic hydrocarbon via catalyzed nonoxidative dehydroaromatization (DHA) are disclosed herein. FIG. 1 depicts some features of the system and process schematically. The system 100 includes a first reaction zone 110 containing a dehydroaromatization catalyst 112. A reactant composition is supplied to the first reaction zone 110. The reactant composition may comprise one or more C₁-C₄ alkanes, including by not limited to methane, ethane, propane, and butane. In one non-limiting embodiment, the reactant comprises natural gas. To simplify the disclosure, FIG. 1 depicts a reactant composition comprising methane. Methane undergoes dehydroaromatization in the presence of catalyst 112 to produce one or more aromatic hydrocarbons. Intermediate compounds are typically produced, such as methane radical (.CH₃) and ethylene (C₂H₄). FIG. 1 depicts the formation of the aromatic hydrocarbons benzene 114 and naphthalene 116.

The dehydroaromatization reaction releases hydrogen. A hydrogen separation membrane 118 is disposed between the reaction zone 110 and a hydrogen stream exit 120. Thus the reaction zone 110 is on the retentate side of the membrane 118 and the hydrogen stream exit 120 is on the permeate side of the membrane. The hydrogen separation membrane 118 selectively removes hydrogen produced in the reaction zone 110. FIG. 1 depicts the hydrogen separation membrane 118 disposed on a porous substrate 122. A porous substrate may be advantageous depending upon the strength and thickness of the membrane 118.

Dehydroaromatization catalysts 112 are known. Some suitable catalysts are metal/zeolite catalysts based on HZSM-5 zeolites. Several different metals have been proposed, including molybdenum, tungsten, rhenium, vanadium, and zinc, with the HZSM-5 zeolites. The rhenium exchanged zeolite (Re/ZSM-5) catalyst is a presently preferred dehydroaromatization catalyst because Re-based H-ZSM5 systems are superior in reactivity, selectivity and stability than the Mo-based systems.

The hydrogen separation membrane 118 is a ceramic membrane that selectively transports H⁺ ions at dehydroaromatization operating temperatures. A variety of metallic, ceramic and polymer membranes have been used for H₂ separation from gas streams. The most common metallic membrane materials are palladium (Pd) and palladium alloys. However, these materials are expensive, strategic and less suitable for H₂ separation from dehydroaromatization reaction since Pd promotes coking. A number of organic membranes (e.g. Nafion® a registered mark of the Dupont Corporation) have also been identified as protonic conductors, but these are limited to lower temperature applications (less than 150° C.). The invention preferably uses a ceramic hydrogen separation membrane 118 that can withstand operation temperatures under a wide range of high-temperatures and that are suitable for promoting the DHA reaction. In some non-limiting embodiments, the hydrogen separation membrane 118 is thermally stable and effective at a temperature above 800° C. In some non-limiting embodiments, the hydrogen separation membrane 118 selectively transports H⁺ ions under a hydrogen partial pressure gradient, a concentration gradient, or an applied voltage. Non-limiting examples of materials from which the hydrogen separation membrane 118 is fabricated include a perovskite, a doped cerate, a doped zirconate, or an acidic phosphate. In one non-limiting embodiment, the hydrogen separation membrane 118 comprises barium. In another non-limiting embodiment, the membrane 118 comprises cerate. In another non-limiting embodiment, the membrane 118 is a composite comprising BaCeO₃ and an electronic conducting phase. The membrane may comprise a 10-30 μm pinhole-free dense membrane.

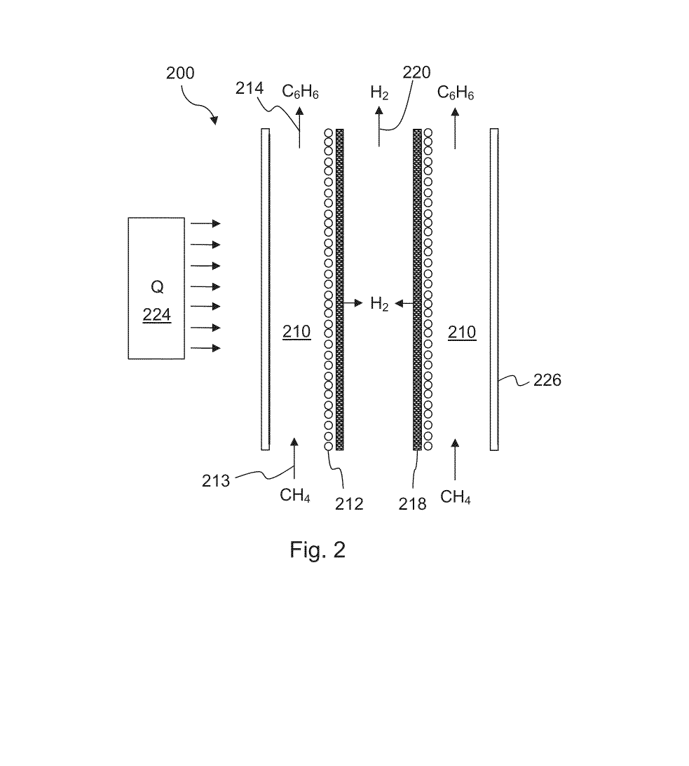

FIG. 2 illustrates one non-limiting system to produce an aromatic hydrocarbon via catalyzed nonoxidative dehydroaromatization. The system 200 includes a first reaction zone 210 containing a dehydroaromatization catalyst 212. A reactant composition 213 is supplied to the first reaction zone 210. The reactant composition 213 may comprise one or more C₁-C₄ alkanes, including but not limited to methane, ethane, propane, and butane. In one non-limiting embodiment, the reactant composition comprises natural gas. FIG. 2 depicts a reactant composition 213 comprising methane. Methane undergoes dehydroaromatization in the presence of catalyst 212 to produce one or more aromatic hydrocarbons 214. For simplicity, FIG. 2 depicts the formation of benzene (C₆H₆) as the aromatic hydrocarbon 214. Other aromatic hydrocarbons may be produced, including but not limited to, toluene, ethylbenzene, styrene, xylene or naphthalene. The dehydroaromatization reaction may also produce a benzene precursor, such as ethylene.

The dehydroaromatization reaction of methane releases hydrogen according to the reaction, 6CH₄→C₆H₆+9H₂. A hydrogen separation membrane 218 is disposed between the reaction zone 210 and a hydrogen stream exit 220. A vacuum or negative pressure may be applied to the hydrogen stream exit 220 to facilitate hydrogen removal. The pressure differential may also facilitate hydrogen transporting across the hydrogen separation membrane 218 from the first reaction zone 210 to the hydrogen stream exit 220.

A heater 224 may be provided to control and maintain the reaction zone 210 at a suitable dehydroaromatization temperature. The dehydroaromatization reaction typically occurs at a temperature in the range from about 500° C. to 1000° C. In some embodiments, the dehydroaromatization reaction occurs at a temperature in the range from about 700° C. to 900° C.

The system 200 depicted in FIG. 2 shows a center hydrogen stream exit 220 surrounded by the reaction zone 210. This configuration may be constructed using concentric tubes, with the center tube being fabricated of a ceramic hydrogen separation membrane material and the outer tube being fabricated of a suitable temperature resistant and inert material. Alternatively, the configuration shown in FIG. 2 may be constructed of parallel plates with suitable sidewalls and seals to form the first reaction zone 210 and hydrogen stream exit 220. While, FIG. 2 depicts the dehydroaromatization catalyst 212 disposed in close proximity to the hydrogen separation membrane 218, it is to be understood that the relative sizes and distances shown in FIG. 2 are for illustration only. In some non-limiting embodiments, the catalyst may substantially fill the reaction zone 210. In other non-limiting embodiments, the outer walls 226 may be disposed close to the catalyst.

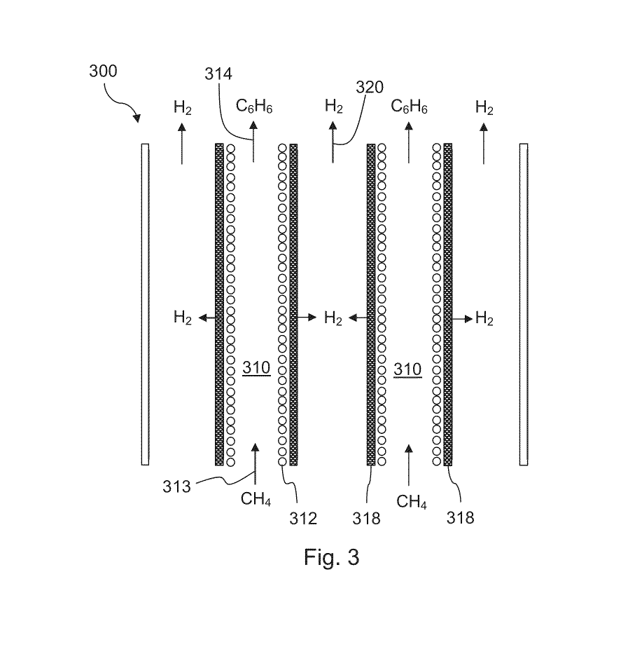

Many alternative configurations may be utilized which combine a dehydroaromatization catalyst and hydrogen separation membrane. FIG. 3 depicts another non-limiting system to produce an aromatic hydrocarbon via catalyzed nonoxidative dehydroaromatization. The system 300 of FIG. 3 is a variation of the system 200 of FIG. 2 and not all common features are illustrated and discussed below. The system 300 includes a first reaction zone 310 containing a dehydroaromatization catalyst 312. A reactant composition 313 is supplied to the reaction zone 310. FIG. 3 depicts a reactant composition 313 comprising methane. Methane undergoes dehydroaromatization in the presence of catalyst 312 to produce one or more aromatic hydrocarbons 314. The dehydroaromatization reaction of methane releases hydrogen. A hydrogen separation membrane 318 is disposed between the reaction zone 310 and a hydrogen stream exit 320. As mentioned above, the reaction zone 310 is on the retentate side of the membrane 318 and the hydrogen stream exit 320 is on the permeate side of the membrane.

The system 300 depicted in FIG. 3 shows multiple first reaction zones 310, multiple hydrogen separation membranes 318, and multiple hydrogen stream exits 320. It will be appreciated that even more first reaction zones 310, combined with hydrogen separation membranes 318 and hydrogen stream exits 320, may be included in alternative systems. Such configurations may be constructed of stacked parallel plates with suitable sidewalls and seals to form the disclosed reaction zones 310 and hydrogen stream exits 320.

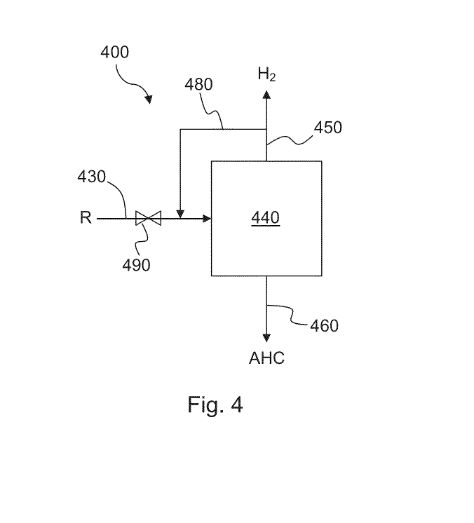

FIG. 4 shows a schematic representation of a non-limiting system 400 to produce an aromatic hydrocarbon (AHC) via catalyzed nonoxidative dehydroaromatization. The systems disclosed in FIGS. 1-3 discussed above, as well as modifications and variations within the level of skill in the art, may be utilized in the system 400 shown in FIG. 4. A reactant feed stream 430 supplies a reactant composition (R) to a reactor 440. The reactor 440 includes one or more reaction zones dehydroaromatization catalyst as disclosed above. The reactor 440 also includes one or more hydrogen separation membranes which enable continuous removal of hydrogen from the reaction zone(s). A hydrogen stream exit 450, which may provide collection of hydrogen from multiple reaction zones, allows for removal and recovery of hydrogen produced during the dehydroaromatization reaction. A product stream exit 460 removes the aromatic hydrocarbon (AHC) produced by the nonoxidative dehydroaromatization of the reactant composition (R) from the reactor 440. A hydrogen recycle stream 480 diverts a portion of hydrogen from the hydrogen stream exit 450 and adds the portion of hydrogen to the reactant composition (R) supplied to the reactor 440.

The hydrogen may also be used to regenerate the dehydroaromatization catalyst. As hydrogen is removed from reactant composition, the resulting hydrocarbon becomes more carbon-rich until coke is formed on the catalyst. Coke deactivates the catalyst. The catalyst may be regenerated by exposing the coke with hydrogen and forming methane according to the following reaction: C+2H₂→CH₄. Catalyst regeneration may be achieved by closing the supply of reactant composition to the reactor with valve 490 and instead supplying hydrogen via the recycle stream 480. To enable continuous operation multiple systems 400 may be used in parallel or series such that while one system is stopped to regenerate the catalyst, other systems may continue operation uninterrupted.

The aromatic products derived from the aforementioned processes may be intermediates to subsequent reactions. For example, benzene can be produced from natural gas in a single step conversion processes via the dehydroaromatization (DHA) route in the absence of oxygen as follows:

6CH₄→C₆H₆+9H₂

The reaction also produces naphthalene and to a limited extent ethylene. The process suffers from limited equilibrium conversion at high temperature (12% conversion at 700° C.). Overcoming the equilibrium limitation requires continuous separation of hydrogen at the reaction temperature. Nearly 100% single pass conversion can be enabled with complete removal of hydrogen. Further, the separated hydrogen can be utilized as a feedstock for subsequent hydrogenation reactions.

In one embodiment, benzene conversion to cyclohexane is carried out via hydrogenation over a nickel (Ni), cobalt (Co) or precious metal catalyst supported on Alumina or similar support. The reaction proceeds as follows:

C₆H₆+3H₂→C₆H₁₂

Suitable precious metal catalysts include palladium and platinum. The catalyst may be in the form of oxidized forms such as PtO₂.

The hydrogenation reaction is simple. It requires a hydrogen source however. It is costly to produce hydrogen as it requires a steam reformer for conversion from methane. Methane is converted syngas (a mixture of CO and H₂). Thus, the production of hydrogen often leads to CO₂ emissions from the carbon content in methane. It would, therefore, be beneficial to have a supply of hydrogen that is not produced from a steam reformer. Hydrogen produced from the dehydroaromitization processes discussed above can be used as a substitute for the reformer source of hydrogen.

Thus, in one embodiment, a method of producing one or more saturated, cyclic hydrocarbons is disclosed. The method includes conversion of natural gas to benzene and naphthalene in a catalyst-membrane reactor; separating hydrogen from the reaction mixture at the temperature of the reaction (700-900° C.) to significantly increase the single pass conversion; adding separated hydrogen in a separate reactor wherein benzene from the first reactor is fed over a catalyst; or adding the separated hydrogen in a separate reactor wherein naphthalene from the first reactor is fed over a catalyst.

In some embodiments, two reactors in series wherein effluent (benzene and naphthalene) from the first reactor is fed to the second reactor and the membrane separated hydrogen from first reactor is fed to the second reactor. In some embodiments, the two reactors are in series. In some embodiments, the two reactions can occur simply in a single reactor with two reaction zones wherein the first reaction zone converts natural gas to benzene and naphthalene and comprises a membrane to separate hydrogen. The second reaction zone contains a hydrogenation catalyst to convert benzene to cyclohexane and naphthalene to decalin.

The second reactor (or reaction zone) converts benzene to cyclohexane and naphthalene to decalin over a hydrogenation catalyst such as nickel, cobalt or a precious metal.

In some embodiments, the conversion of benzene to cyclohexane and conversion of naphthalene to decalin can be carried out in the same reactor or two different reactors.

In some embodiments, the unreacted natural gas (methane) is separated and recycled back to the first reaction zone.

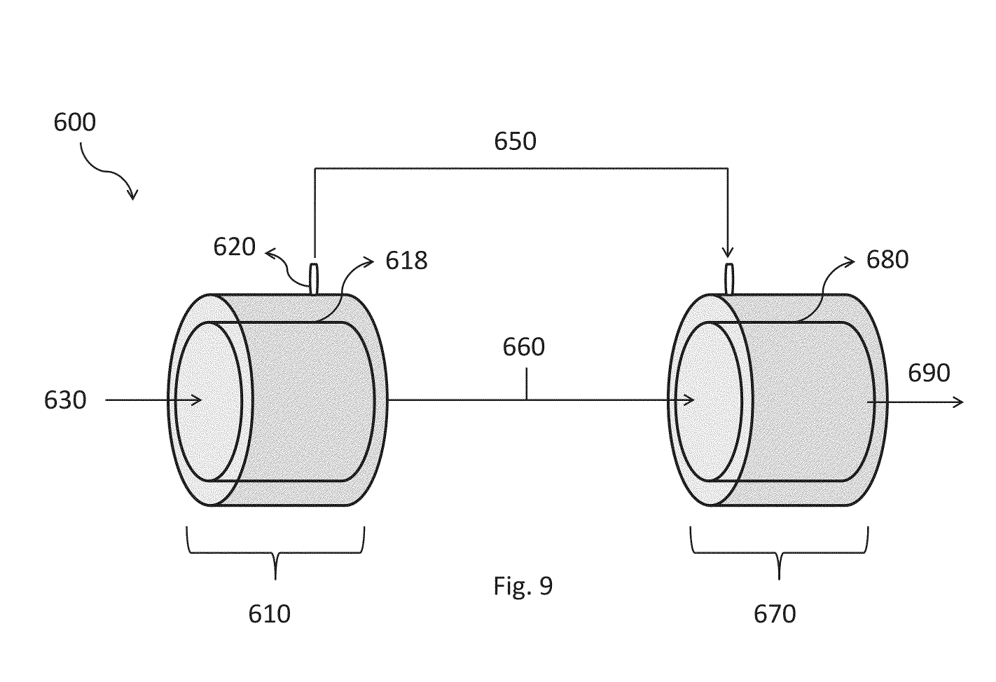

FIG. 9 depicts some features of the system and process schematically. The system 600 includes a first reaction zone 610 containing a dehydroaromatization catalyst 612. A reactant composition is supplied to the first reaction zone 610 via an inlet 630. The reactant composition may comprise one or more C₁-C₄ alkanes, including by not limited to methane, ethane, propane, and butane. In one non-limiting embodiment, the reactant comprises natural gas. Methane undergoes dehydroaromatization in the presence of catalyst 612 to produce one or more aromatic hydrocarbons. Intermediate compounds are typically produced, such as methane radical (.CH₃) and ethylene (C₂H₄).

The dehydroaromatization reaction releases hydrogen. A hydrogen separation membrane 618 is disposed between the reaction zone 610 and a hydrogen stream exit 620. In some embodiments, the reaction zone 610 is on the retentate side of the membrane 618 and the hydrogen stream exit 620 is on the permeate side of the membrane. The hydrogen separation membrane 618 selectively removes hydrogen produced in the reaction zone 610.

Aromatic hydrocarbon intermediates produced by the dehydroaromatization occurring in the first reaction zone 610 are removed through a hydrocarbon transfer conduit 660 and introduced into the second reaction zone 670. The second reaction zone includes a hydrogenation catalyst 680. Hydrogen gas removed from the first reaction zone 610 at hydrogen stream exit 620 is passed through a hydrogen transfer conduit 650 and into the second reaction zone 670. Upon contact with the aromatic hydrocarbon intermediate and hydrogen catalyst 680, the hydrogen gas reduces the aromatic hydrocarbon to a saturated, cyclic hydrocarbon as effluent stream 690.

In some embodiments, a separator may be used to separate the different aromatic intermediates present in the effluent of the first reaction zone. For example, benzene may be separated from naphthalene using a flash drum or other flash evaporation. In some embodiments, a separator may be used to separate the unreacted hydrocarbon (e.g. methane) from reacted hydrocarbon (e.g. benzene). The separator may, therefore, serve the purpose of enabling recycling of unreacted feed stream

In some embodiments, a separator may be used to separate the different saturated, cyclic hydrocarbons present in the effluent of the second reaction zone. For example, cyclohexane may be separated from decalin using a distillation column.

The following examples are given to illustrate various embodiments within, and aspects of, the present disclosure. These are given by way of example only, and it is understood that the following examples are not comprehensive or exhaustive of the many types of embodiments of the present invention that can be prepared in accordance with practicing this disclosure.

### Example 1

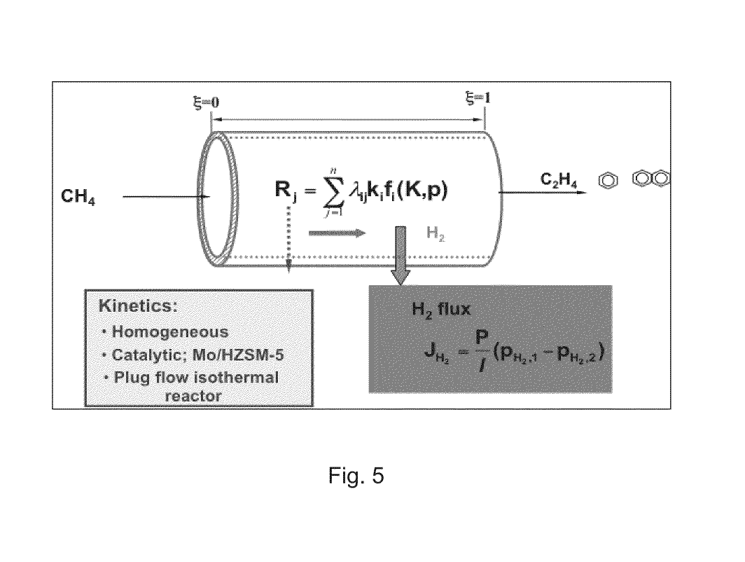

Computer chemical reaction simulation studies were performed to model and examine the operation of the dehydroaromatization reaction using the combination of a dehydroaromatization catalyst with a hydrogen separation membrane for continuous hydrogen removal from the reaction zone. Chemical reaction simulation software, CHEMKIN®, provided by Reaction Design, San Diego, Calif. was used to perform the chemical reaction simulation studies. The CHEMKIN® model and simulations methodology is shown as a schematic in FIG. 5.

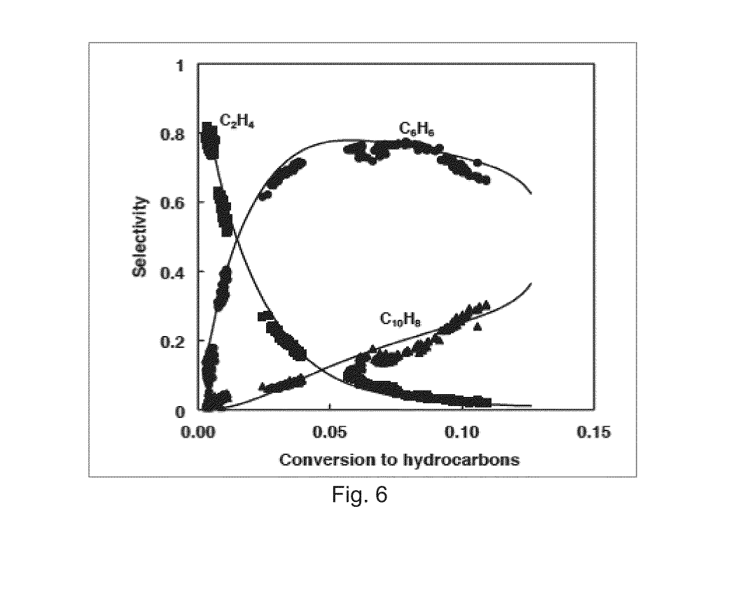

The CHEMKIN® chemistry simulation results suggest that bi-functional catalysts, such as Metal/H-ZSM5, with continuous H₂ removal provide almost complete CH₄ conversion at practical residence time (100 s) and intermediate values of dimensionless transport rates (ratio of permeation to reaction of 1-10. For currently available hydrogen separation membrane materials, such values suggest a membrane thickness less than 100 μm of dense ceramic material. The model results also mapped appropriately with experimental data as shown in FIG. 6 which graphically presents experimental results verses chemical model simulated product selectivities as a function of methane conversion, which was varied by changing the reactor residence time at constant temperature (950 K) and inlet methane partial pressure (0:5 bar) and by allowing the number of sites within the reactor to decrease as deactivation occurs.

### Example 2

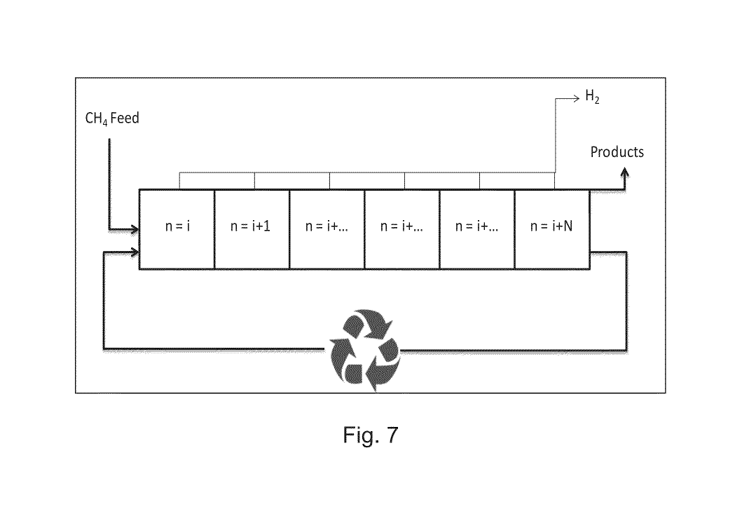

Chemical process simulation software, Aspen® Plus, provided by Aspen Technology, Inc. was used to evaluate methane conversion as a function of percentage hydrogen removal. A dehydroaromatization reactor having a hydrogen separation membrane was simulated using Aspen® Plus in order to approximate the yield of benzene production and methane conversion. Six equilibrium reactors were used in series segregated by separator blocks that were used to approximate the removal of methane. Each reactor was coupled with a separator to simulate a “node” in the membrane reactor, thereby discretizing the reactor. A recycle loop stream was also included in the system allowing for unwanted products to be suppressed in the reactor. A conceptual model of the simulation can be found in FIG. 7.

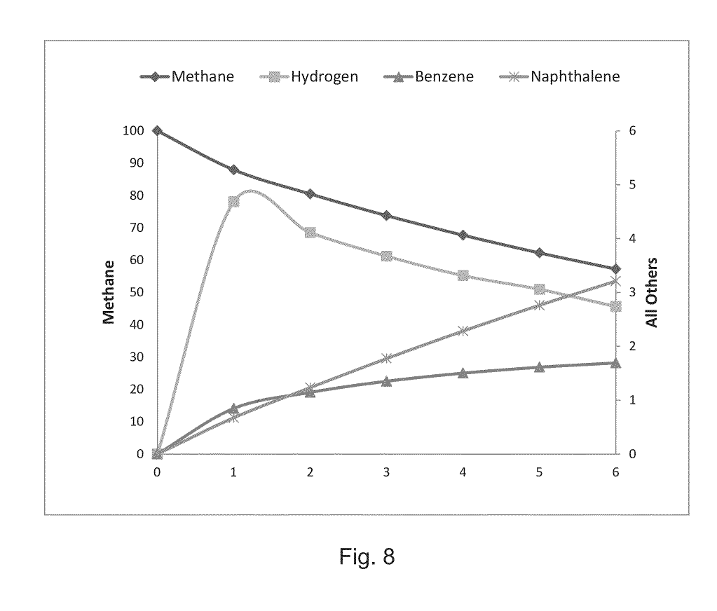

Two different reactions were modeled in this simulation (the dehydration of methane to form benzene and for the formation of naphthalene). The previous stoichiometric equations do not take into account the intermediate products that would be involved with these reactions. This is justified, because it was assumed that each node comes to thermodynamic equilibrium as calculated by Gibb's minimization, wherein only the products and reactants are of concern. By assuming that each node comes to thermodynamic equilibrium, it is also implicitly assumed that the reactions are either infinitely fast or the node is of infinite volume, as well as perfect mixing in the reactor. FIG. 8 shows the results of simulations. It can be observed that 60% methane conversion is possible with removal of a fraction of hydrogen.

### Example 3

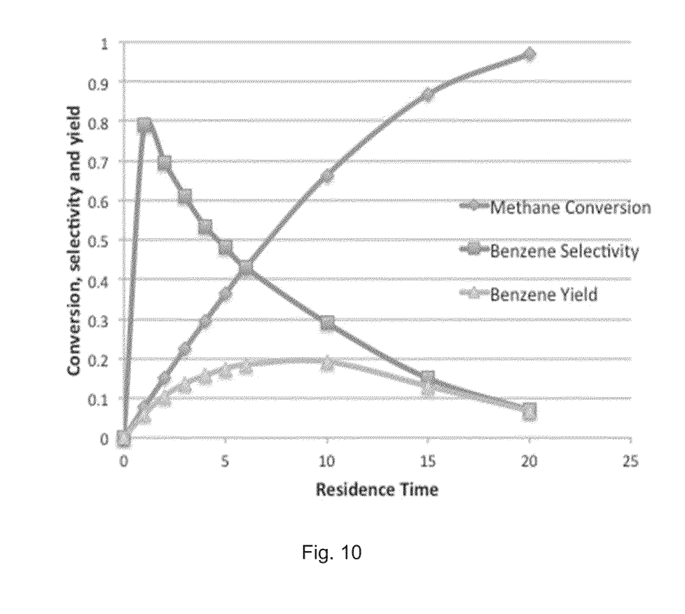

FIG. 10 is a graph showing the results of a chemical process simulation for conversion of methane to aromatic hydrocarbon intermediates with benzene selectivity and yield as a function of hydrogen removal. Reactor and process modeling was performed using Matlab (version R2013b) and Aspen Plus (version 8.0), respectively. The reactor scheme in Matlab consists of isothermal differential plug flow reactor equations describing a set of three equilibrium reactions.

2CH₄ [symbol] C₂H₄+2H₂

3C₂H₄ [symbol] C₆H₆+3H₂

C₆H₆+2C₂H₄ [symbol] C₁₀H₈+3H₂

The reactor simulations are carried out at 973 K and 1.01 bar with reactor dimensions of 1.47 cm radius and 1.8 cm length. Inputs to the simulation are the feed composition (85% methane, 15% argon) and flow (27.3 sccm), hydrogen removal, equilibrium constants, and reaction rate constants.

Equilibrium constants for the reactions above were found using a Gibbs minimization reactor in Aspen Plus. The same feed and reactor conditions are entered to Aspen as are entered to Matlab and the simulation is carried out isothermally. Aspen will calculate phase and chemical equilibrium such that Gibbs energy is minimized and return the results as the reactor outlet stream. This stream composition is then used to calculate the equilibrium constant K₁ for each reaction i as

$${K}_{i} = \frac{{{[ C ]}^{a} [ D ]}^{b}}{{{[ A ]}^{c} [ B ]}^{d}}$$

where the bracketed term is the species concentration in mol/cm³, and the exponent is its corresponding stoichiometric coefficient in reaction i. Since Aspen will not automatically return the species concentrations but will rather report species molar flow rates, concentrations were found by dividing the molar flow rate by the outlet volumetric flow rate. Furthermore, the equilibrium methane conversion was found by

$$\frac{( moles {CH}_{4} in ) - ( moles {CH}_{4} out )}{moles {CH}_{4} in} .$$

This procedure was carried out over a range of temperatures to evaluate the effect of temperature on the equilibrium constant and equilibrium methane conversion.

The results of this Aspen Gibbs minimization were confirmed using the van't Hoff equation (below) with calculation steps and variable definitions detailed elsewhere.

$$ln ( \frac{{K}_{{T}_{2}}}{{K}_{{T}_{1}}} ) = \frac{1}{R} {\int }_{{T}_{1}}^{{T}_{2}} \frac{Δ {h}^{o}}{{T}_{2}} d T$$

Equilibrium constants from Aspen were fed into Matlab along with experimentally calculated reaction rates. The hydrogen removal parameter, k_{b}, was adjusted by trial and error to meet the target of 30% methane conversion. A hydrogen balance around the reactor reveals the quantity of hydrogen removed, and given the reactor dimensions, allows the hydrogen flux to be calculated as 0.344 μmol/(cm²*s).

Based on the conversions and product composition determined by Matlab, fractional conversions for each reaction in the process simulation (Aspen), as well as hydrogen removal, were adjusted to produce the same results as Matlab. In this way, the Matlab results form the basis for the Aspen inputs and the two models are linked.

While specific embodiments and examples of the present invention have been illustrated and described, numerous modifications come to mind without significantly departing from the spirit of the invention, and the scope of protection is only limited by the scope of the accompanying claims.

1. An apparatus to produce one or more cyclic, saturated hydrocarbons, comprising:
a first reaction zone comprising a dehydroaromatization catalyst;
a second reaction zone comprising a hydrogenation catalyst;
a first inlet that provides a reactant hydrocarbon to the first reaction zone;
a heater for heating the first reaction zone;
a hydrocarbon transfer conduit located between the first and second reaction zones;
a hydrogen transfer conduit located between the first and second reaction zones; and
a hydrogen separation membrane disposed between the first reaction zone and the hydrogen transfer conduit.

2. The apparatus of claim 1, wherein the first and second reaction zones are located in a single reactor.

3. The apparatus of claim 1, wherein the first and second reaction zones are located in separate reactors.

4. The apparatus of claim 1, wherein the hydrogenation catalyst is nickel or cobalt.

5. The apparatus of claim 1, further comprising a hydrocarbon separator capable of separating reactant hydrocarbon from the first reaction zone.

6. The apparatus of claim 1, further comprising a vacuum means operably connected to the hydrogen separation membrane and hydrogen transfer conduit.

7. The apparatus of claim 1, wherein the hydrogen separation membrane comprises a ceramic membrane that selectively transports H⁺ ions at dehydroaromatization operating temperatures.

8. The apparatus of claim 1, wherein the hydrogen separation membrane selectively transports H⁺ ions under a hydrogen partial pressure gradient, a concentration gradient, or an applied voltage.

9. The apparatus of claim 1, wherein the hydrogen separation membrane comprises a perovskite, a doped cerate, a doped zirconate, or an acidic phosphate.

10. The apparatus of claim 1, wherein the hydrogen separation membrane comprises a Ba-cerate ceramic composite

11. The apparatus of claim 1, wherein the reactant comprises methane.

12. A method of preparing one or more cyclic, saturated hydrocarbons, comprising:
contacting a reactant hydrocarbon with a dehydroaromatization catalyst in a first reaction zone to produce one or more aromatic intermediates;
heating the first reaction zone;
removing hydrogen from the first reaction zone through a hydrogen separation membrane;
transferring the aromatic intermediate from the first reaction zone to the second reaction zone;
providing the separated hydrogen to the second reaction zone; and
contacting the aromatic intermediate with a hydrogenation catalyst in a second reaction zone resulting in one or more cyclic, saturated hydrocarbons.

13. The method of claim 13, wherein the first and second reaction zones are located in a single reactor.

14. The method of claim 13, wherein the first and second reaction zones are located in separate reactors.

15. The method of claim 13, wherein the hydrogenation catalyst is nickel or cobalt.

16. The method of claim 13, wherein the hydrogen separation membrane comprises a ceramic membrane that selectively transports H⁺ ions at dehydroaromatization operating temperatures.

17. The method of claim 13, wherein the hydrogen separation membrane selectively transports H⁺ ions under a hydrogen partial pressure gradient, a concentration gradient, or an applied voltage.

18. The method of claim 13, wherein the hydrogen separation membrane comprises a perovskite, a doped cerate, a doped zirconate, or an acidic phosphate.

19. The method of claim 13, wherein the hydrogen separation membrane comprises a Ba-cerate ceramic composite.

20. The method of claim 13, wherein the reactant hydrocarbon comprises methane.

21. The method of claim 13, further comprising periodically regenerating the dehydroaromatization catalyst by contacting the dehydroaromatization catalyst with hydrogen.

22. The method of claim 13, further comprising separating cyclic, hydrocarbons from one another.

23. The method of claim 13, wherein the one or more cyclic, hydrocarbons includes decalin.

24. The method of claim 13, wherein the one or more cyclic, hydrocarbons includes cyclohexane.
